(19) United States
(12) Patent Application Publication
(10) Pub. No.: US 2007/0144529 A1
(43) Pub. Date: Jun. 28, 2007

(54) Dimensionally expanding condom

(76) Inventor: Latrill Bryant, Baltimore, MD (US)

Correspondence Address: Ober, Kaler, Grimes & Shriver;Royal W. Craig, 120 East Baltimore Street, Baltimore, MD, 21202-1643, US

(21) Appl. No.: 11/646,137

(22) Filed: Dec. 26, 2006

Related U.S. Application Data
(60) Provisional application No. 60/753,544, filed on Dec. 23, 2005.

(51) Int. Cl. A61F 6/04

(52) U.S. Cl. 128/844

(57) ABSTRACT

The device of the present invention provides for a prophylactic and prosthetic condom to be worn by a male person which can add to the size of the male sexual organ. The condom is constructed to have a two-layer tubular sheath member which is formed of a suitable elastic material such as latex. It has an open end for receiving a penis therein and an opposite closed end. Between the two layers of the sheath is disposed a chemically reactive expandable material. The expandable material reacts to a chemical reactant which is introduced by the user or by some other suitable means. When packaged for sale, the present invention will be as compact as an ordinary condom. When in use, the material inside the condom, by reaction to a chemical, will expand adding to the size of the wearer's penis.

### CROSS-REFERENCE TO RELATED APPLICATION

The present application derives priority from U.S. provisional application No. 60/753,544 filed 23 Dec. 2005.

### BACKGROUND OF THE INVENTION

1. Field of the invention

The present invention relates to prophylactic condoms and, more particularly, to an improved dimensionally expanding condom for temporarily increasing the size of the wearer's penis.

2. Description of the Background

Generally, condoms have served two purposes in the past. They have acted as a means of contraception and as a means to prevent the spread of sexually transmitted diseases.

Recently, several condoms have been invented which serve other purposes as well.

For example, U.S. Pat. No. 5,137,032 teaches a condom which is provided with a friction imparting agent to hold the condom partially in place during intercourse. It also discloses an embodiment wherein the distal closed portion of the condom has a larger diameter than the proximal open portion, to provide further friction for the wearer during intercourse.

U.S. Pat. No. 5,191,902 shows a prophylactic device which is formed of an inner and an outer condom, which are heat sealed together at the bases. The base of the outer condom is provided with several apertures for the release of air between the two condoms. This device however does not provide for the placement of any material between the layers.

In U.S. Pat. No. 4,498,466 the condom disclosed has a slide fastener for easier placement on a flaccid penis. The condom also has a plurality of compartments filled with a rheopexic material which stiffens upon use. This patent also teaches a novel type of penile implant prosthesis formed of the rheopexic material which stiffens upon agitation.

U.S. Pat. No. 4,432,357 provides for a condom having a plurality of sealed chambers along a portion of the shaft of the condom. The chambers are filled with a rheopexic material which react to shear stress thereby stiffening the rheopexic material inside the chambers of the condom.

However, none of these devices disclose a condom which can temporarily increase the size of the wearer's penis.

### SUMMARY OF THE INVENTION

It is, therefore, an object of the present invention to provide a condom which can add to the size of the male sexual organ.

It is another object of this invention to provide for a double layer condom containing a chemically reactive material which expands upon exposure to a suitable substance.

In accordance with the above and other objects, the device of the present invention is a condom comprised of a two-layer tubular sheath member which is formed of a suitable elastic material such as latex. The condom has an open end for receiving a penis therein and an opposite closed end. Between the two layers of the sheath is disposed a suitable chemically reactive expandable material. The expandable material reacts to a chemical-or other reactive substance which is introduced by the user or by some other suitable means. When packaged for sale, the present invention will be as compact as an ordinary condom and is meant to be used one time and thereafter disposed. During use, the material will react and expand, depending upon the positioning of the material between the two sheath layers, along the sides, at the closed end or both; it will add to either the length or the width or both of the male sexual organ.

The present invention performs the normal prophylactic functions of a condom acting as a method of birth control and protecting against sexually transmitted diseases. However, this condom is also an inexpensive, disposable means for adding to the size of the male sexual organ.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a side cross sectional view of a first embodiment of a condom 1 according to the present invention.

FIG. 2 is a side cross sectional view of a second embodiment of a condom 1 according to the present invention.

FIG. 3 is a side cross sectional view of a third embodiment of a condom 1 according to the present invention.

FIG. 4 is a cross sectional view of the condom 1 shown in FIG. 3.

### DETAILED DESCRIPTION OF THE PREFERRED EMBODIMENTS

The present invention is a condom 1 comprised of a two-layer tubular sheath member having an open end 5 for receiving a penis therein and an opposite closed end 6. The tubular sheath member has an inner layer 4 and an outer layer 3 with a suitable chemically reactive expandable material 2 disposed there between.

The outer layer 3 is formed as an elongate tubular sheath with a distal closed end and an opposing open end.

The inner layer 4 is likewise formed as an elongate tubular sheath with a distal closed end and an opposing open end, and the diameter of the inner layer 4 is slightly smaller than that of the outer layer 3 so that the inner layer 4 fits coaxially inside the outer layer 3.

The inner layer 4 is integrally joined to the outer layer 3 at the respective open ends by a seal 9, which may be an integral annular wall running between the two layers 3, 4 to form a closed fluid-tight chamber there between.

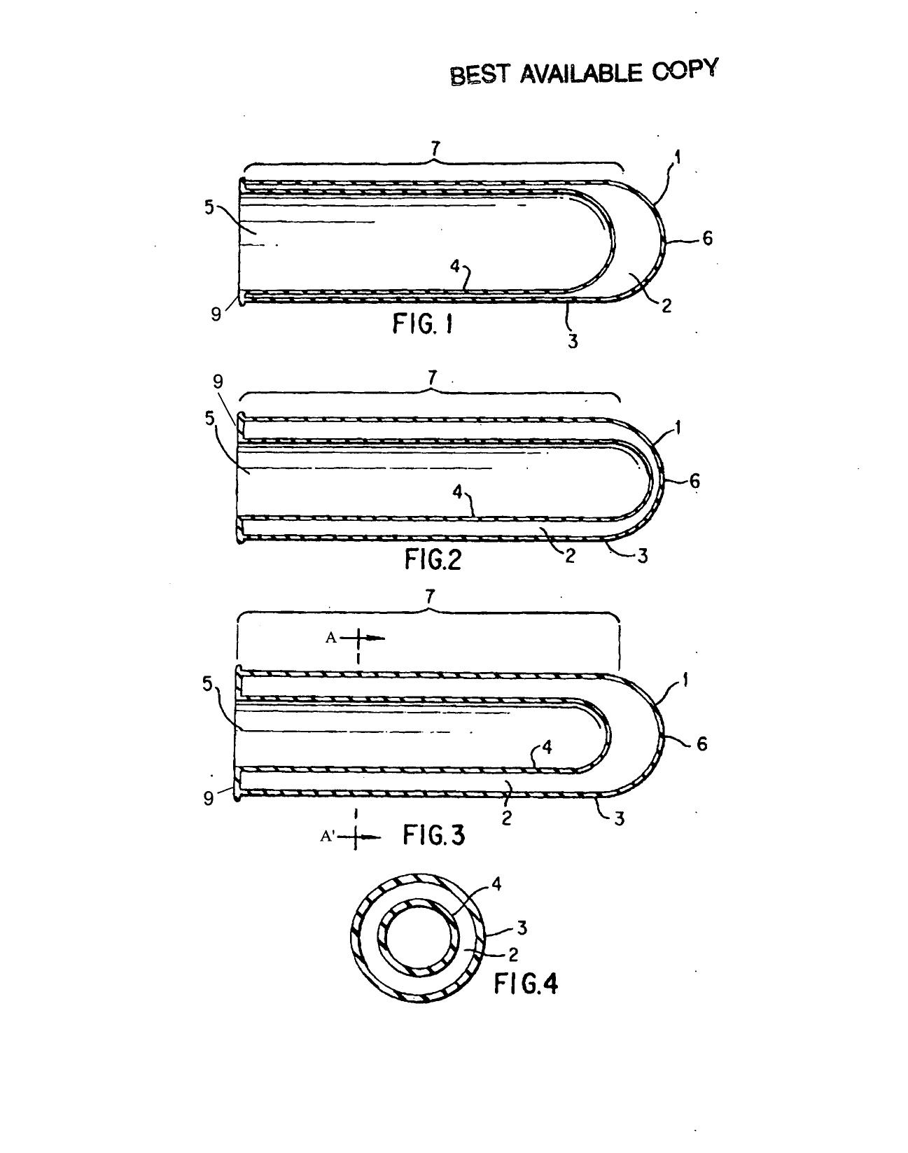

In FIG. 1 the chemically reactive expandable material 2 is positioned between the inner layer 4 and the outer layer 3 at the closed end 6 of the condom 1, so as to-add to the length of the wearer's penis.

In FIG. 2 the chemically reactive expandable material 2 is positioned between the inner layer 4 and the outer layer 3 along the shaft 7 of the condom 1, so as to add to the width of the wearer's penis.

In FIG. 3 the chemically reactive expandable material 2 is positioned between the inner layer 4 and the outer layer 3 both at the closed end 6 and along the shaft 7 of the condom 1, so as to add to the width and length of the wearer's penis.

FIG. 4 shows a cross sectional view of the condom along the A-A′ line of FIG. 3. The inner layer 4 is separated from the outer layer 3 by the chemically reactive expandable material 2.

The chemically reactive expandable material is in a compressed unreacted state when the condom is packaged for sale. A chemical reactant initiates a process whereby the condom expands. The chemically reactive expandable material is exposed to a chemical reactant when unpackaged, thereby causing the chemically reactive expandable material to expand but remain in the position where it has expanded. Consequently, this reaction adds to either the length, width, or both of the wearer's penis. The chemical reactant is introduced by a mechanism such as a bulb holding the chemical reactant and a tube which extends between the two layers 3 and 4 of the condom 1 holding the chemically reactive expandable material 2. When the bulb is squeezed the chemical reactant is forced in through the tube and reacts with the expandable material.

Another mechanism is to provide for a frangible balloon containing a chemical reactant to be incorporated into the expandable material, when the condom is manufactured. The user can squeeze the balloon bursting it and dispensing the chemical reactant throughout the expandable material. Other means can be used as well to accomplish the introduction of a chemical to the expandable material without going beyond the scope of this invention.

The chemically reactive expandable material may include a compressed dried absorbent material, such as an absorbent layer of compressed, cellulose based, linear fibers for expanding the condom in three-dimensions while being worn by a user. The fibers may be completely unbonded or, alternatively, may be bonded with a binder prior to compression. Suitable binders are those that are water soluble; they may be used in such an amount that does not impair the subsequent swelling of the fibers, preferably an amount not greater than 20% in weight. The layer of compressed, cellulose based, linear fibers may be activated by water. Alternatively, a suitable chemically reactive expandable water gel polymer may be used. This is an odorless water-absorbing polymer (polyacrylamide) that, when exposed to water, absorbs the water, expands, and turns into a crystalline gel. The chemically reactive expandable material may comprise any other suitable porous material that expands in contact with moisture.

The chemical reactant may include water, for example, or other non-toxic fluid.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth herein.

What is claimed is:

1. A dimensionally expanding condom for adding to the size of a penis, comprising:
an outer tubular sheath member with a distal closed end and an opposing open end;
an inner tubular sheath member with a distal closed end and an opposing open end, said inner tubular sheath member being dimensioned to fit inside said outer tubular member;
said inner tubular sheath member being integrally joined to the outer tubular sheath member at the respective open ends to form a closed fluid-tight chamber there between;
a chemically reactive expandable material and a chemical reactant disposed between said inner tubular sheath member and said outer tubular sheath member, wherein said chemically reactive expandable material reacts with said chemical reactant and expands, thereby adding to the size of said penis.

2. A condom according to claim 1, wherein said chemically reactive expandable material is positioned between said inner tubular sheath member and said outer tubular sheath member at said closed ends.

3. A condom according to claim 1, wherein said chemically reactive expandable material is positioned between said inner tubular member and said outer tubular member along said shaft portions.

4. A condom according to claim 1, wherein said chemically reactive expandable material is positioned between said inner tubular member and said outer tubular member along said closed ends and said shaft portions.

5. A condom according to claim 1, wherein said outer and inner tubular sheath members comprise an elastic material.

6. A condom according to claim 5, wherein said elastic material comprises latex.

7. A condom according to claim 1, wherein said chemically reactive expandable material is a compressed dried absorbent material.

8. A condom according to claim 7, wherein said chemically reactive expandable material is cellulose.

9. A condom according to claim 1, wherein said chemical reactant is water.

10. A dimensionally expanding condom for adding to the size of a penis comprising:
an outer tubular sheath member having a shaft portion with a distal closed end and an opposing open end;
an inner tubular sheath member having a shaft portion with a distal closed end and an opposing open end joined edgewise to said open end of said outer tubular sheath member;
a chemically reactive expandable material disposed between said inner tubular sheath member and said outer tubular sheath member, wherein said chemically reactive expandable material reacts upon exposure to a chemical reactant and expands, thereby adding to the size of said penis; and
a means for introducing said chemical reactant into said chemically reactive expandable material.

11. A condom according to claim 10, wherein said outer and inner tubular sheath members comprise an elastic material.

12. A condom according to claim 11, wherein said elastic material comprises latex.

13. A condom according to claim 10, wherein said chemically reactive expandable material is a compressed dried absorbent material.

14. A condom according to claim 10, wherein said chemically reactive expandable material is cellulose.

15. A condom according to claim 10, wherein said chemical reactant is water.

16. A dimensionally expanding condom for adding to the size of a penis comprising:
an outer tubular sheath member having a shaft portion with a distal closed end and an opposing open end;
an inner tubular sheath member having a shaft portion with a distal closed end and an opposing open end joined edgewise to said open end of said outer tubular sheath member; a chemically reactive expandable material disposed between said inner tubular sheath member and said outer tubular sheath member, said expandable material includes a balloon comprising a chemical reactant within said balloon, wherein said chemically reactive expandable material reacts with said chemical reactant upon bursting of said balloon, said expandable materials expands and thereby adds to the size of said penis.

17. A condom according to claim 16, wherein said outer and inner tubular sheath members comprise an elastic material.

18. A condom according to claim 16, wherein said chemically reactive expandable material is a compressed dried absorbent material.

19. A condom according to claim 16, wherein said chemically reactive expandable material is cellulose.

20. A condom according to claim 16, wherein said chemical reactant is water.
